(19) United States
(12) Patent Application Publication
(10) Pub. No.: US 2023/0363655 A1
(43) Pub. Date: Nov. 16, 2023

(54) Method and Apparatus for Generating an Electrocardiogram from a Photoplethysmogram

(71) Applicant: Queen's University at Kingston, Kingston (CA)

(72) Inventors: Pritam Sarkar, Kingston (CA); Ali Etemad, Kanata (CA)

(21) Appl. No.: 18/029,260

(22) PCT Filed: Sep. 30, 2021
(86) PCT No.: PCT/CA2021/051368

§ 371 Date: Mar. 29, 2023

Related U.S. Application Data
(60) Provisional application No. 63/085,394, filed on Sep. 30, 2020.

(51) Int. Cl. A61B 5/024; A61B 5/327; A61B 5/00; G06N 3/08

(52) U.S. Cl. CPC A61B 5/02416; A61B 5/327; A61B 5/7267; G06N 3/08

(57) ABSTRACT

Electrocardiogram (ECG) is the electrical measurement of cardiac activity, whereas photoplethysmogram (PPG) is the optical measurement of volumetric changes in blood circulation. While both signals are used for heart rate monitoring, from a medical perspective, ECG is more useful as it carries additional cardiac information. For continuous cardiac monitoring, PPG sensors are practical. Methods for generating an ECG from a PPG signal may include subjecting the PPG signal to a deep learning network trained to generate a corresponding ECG. The deep learning network may include an adversarial model such as a generative adversarial network (GAN) that may use an attention-based generator to learn local salient features, and may also use dual discriminators to preserve the integrity of generated data in both time and frequency domains

### FIELD

The invention relates to methods and apparatus for generating ECG signals from PPG signals using techniques based on trained deep learning networks. The deep learning networks may include adversarial models such as a generative adversarial network.

### BACKGROUND

According to the World Health Organization (WHO) in 2017, Cardiovascular Deceases (CVDs) are reported as the leading causes of death worldwide (WHO 2017). The report indicates that CVDs cause 31% of global deaths, out of which at least three-quarters of deaths occur in the low or medium-income countries. One of the primary reasons behind this is the lack of primary healthcare support and the inaccessible on-demand health monitoring infrastructure. Electrocardiogram (ECG) is considered as one of the most important attributes for continuous health monitoring required for identifying those who are at serious risk of future cardiovascular events or death. A vast amount of research is being conducted with the goal of developing wearable devices capable of continuous ECG monitoring and feasible for daily life use, largely to no avail. Currently, very few wearable devices provide wrist-based ECG monitoring, and those that do require the user to stand still and touch the watch with both hands in order to close the circuit in order to record an ECG segment of limited duration (usually 30 seconds), making these solutions non-continuous and sporadic.

Photoplethysmogram (PPG), an optical method for measuring blood volume changes under the skin, is considered as a close alternative to ECG, which contains some cardiovascular information such as heart rate. Moreover, through recent advancements in smartwatches, smartphones, and other similar wearable and mobile devices, PPG has become the industry standard as a simple, wearable-friendly, and low-cost solution for continuous heart rate (HR) monitoring for everyday use. Nonetheless, PPG suffers from inaccurate HR estimation and several other limitations in comparison to conventional ECG monitoring devices (Bent et al. 2020) due to factors like skin tone, diverse skin types, motion artefacts, and signal crossovers among others. Moreover, the ECG waveform carries important information about cardiac activity. For instance, the P-wave indicates the sinus rhythm, whereas a long PR interval is generally indicative of a first degree heart blockage (Ashley and Niebauer 2004). As a result, ECG is consistently being used by cardiologists for assessing the condition and performance of the heart.

As to PPG-to-ECG translation, Zhu et al. (2019b) used a discrete cosine transformation (DCT) technique to map each PPG cycle to its corresponding ECG cycle. First, onsets of the PPG signals were aligned to the R-peaks of the ECG signals, followed by a de-trending operation in order to reduce noise. Next, each cycle of ECG and PPG was segmented, followed by temporal scaling using linear interpolation in order to maintain a fixed segment length. Finally, a linear regression model was trained to learn the relation between DCT coefficients of PPG segments and corresponding ECG segments. In spite of several contributions, this study suffers from several limitations. First, the model failed to produce a reliable ECG in a subject-independent manner, which limits its application to only previously seen subject's data. Second, often the relation between PPG segments and ECG segments are not linear, therefore in several cases, this model failed to capture the non-linear relationships between these two domains. Lastly, no experiments have been performed to indicate any performance enhancement gained from using the generated ECG as opposed to the available PPG (for example a comparison of measured HR).

### SUMMARY

Described herein are methods, apparatus, and structures (e.g., software) for generating ECG signals from input PPG signals. Embodiments may aid with continuous and reliable cardiac monitoring. One embodiment may use PPG segments to generate corresponding ECG segments of equal length. Machine learning techniques, such as a deep neural network, e.g., a generative adversarial network, may be used to learn mapping between PPG and ECG signals. Self-gated soft-attention may be used in a generator to learn selected regions of ECG waveforms (i.e., selected from among PQRSTU regions), for example the QRS complex. Embodiments may use a dual discriminator strategy to learn mapping in both time and frequency domains.

One aspect of the invention relates to a method for generating an ECG signal from a corresponding PPG signal, comprising: receiving a PPG of a subject; subjecting the PPG to a deep learning network trained to generate a corresponding ECG; and outputting the generated ECG.

In one embodiment, the deep learning network comprises a generative adversarial network (GAN) trained using unpaired PPG and ECG signals; wherein the unpaired signals are obtained: (a) from the same subject at different times; or (b) from different subjects.

In one embodiment, the deep learning network comprises a generative adversarial network (GAN) trained using paired PPG and ECG signals; wherein the paired signals are obtained from the same subject at the same time.

In one embodiment, the GAN comprises at least one generator and at least one discriminator.

In one embodiment, the at least one discriminator operates on ECG signals in the time domain.

In one embodiment, the GAN comprises at least one generator and first and second discriminators; wherein the at least one generator translates the PPG to an ECG signal; wherein the first discriminator operates on ECG signals in the frequency domain; and wherein the second discriminator operates on ECG signals in the time domain.

In one embodiment, the GAN comprises first and second generators and first to fourth discriminators; herein the first generator translates the PPG to an ECG; wherein the second generator translates the ECG to PPG; wherein the first and second discriminators operate on ECG signals in the frequency and time domains, respectively; and wherein the third and fourth discriminators operate on ECG signals in the frequency and time domains, respectively.

In one embodiment, at least one generator is an attention-based generator.

In one embodiment, the attention-based generator focusses on at least one selected region of the PPG and the generated ECG.

In one embodiment, the selected region comprises one or more of a P, Q, R, S, T, U component of the generated ECG.

One embodiment comprises estimating heart rate (HR) using the generated ECG and the input PPG.

In one embodiment, the method as described herein is implemented in an electronic device.

In one embodiment, the electronic device is wearable.

Another aspect of the invention relates to an electronic device, comprising: a processor that receives PPG signal as an input; wherein the processor implements a deep learning network trained to generate an ECG from the PPG; and an output device connected to the processor that outputs the generated ECG signal.

Another aspect of the invention relates to an electronic device, comprising: a PPG sensor that obtains PPG signal of a subject; a processor that receives the PPG as an input; wherein the processor implements a deep learning network trained to generate an ECG from the PPG; and an output device connected to the processor that outputs the generated ECG.

In one embodiment, the electronic device is adapted to be worn by a subject; wherein the PPG sensor obtains PPG of the subject; wherein the output generated ECG is based on the subject's PPG.

Another aspect of the invention relates to non-transitory computer readable media for use with a processor, the computer readable media having stored thereon instructions that direct the processor to: receive PPG of a subject; implement a deep learning network; subject the PPG to the deep learning network to generate a corresponding ECG; and output the generated ECG.

In one embodiment of the non-transitory computer readable media, the deep learning network comprises a generative adversarial network (GAN).

In one embodiment of the non-transitory computer readable media, the GAN comprises at least one generator and at least one discriminator.

In one embodiment of the non-transitory computer readable media, the at least one discriminator operates on ECG signals in the time domain.

In one embodiment of the non-transitory computer readable media, the GAN comprises at least one generator and first and second discriminators; wherein the at least one generator translates the PPG to an ECG; wherein the first discriminator operates on ECG signals in the frequency domain; and wherein the second discriminator operates on ECG signals in the time domain.

In one embodiment of the non-transitory computer readable media, the GAN comprises first and second generators and first to fourth discriminators; wherein the first generator translates the PPG to an ECG; wherein the second generator translates the ECG to PPG; wherein the first and second discriminators operate on ECG signals in the frequency and time domains, respectively; and wherein the third and fourth discriminators operate on ECG signals in the frequency and time domains, respectively.

In one embodiment of the non-transitory computer readable media, at least one generator is an attention-based generator.

In one embodiment of the non-transitory computer readable media, the attention-based generator focusses on at least one selected region of the PPG and the generated ECG.

In one embodiment of the non-transitory computer readable media, the selected region comprises one or more of a P, Q, R, S, T, U component of the generated ECG.

In one embodiment of the non-transitory computer readable media, the instructions direct the processor to estimate heart rate using the generated ECG and the input PPG.

### BRIEF DESCRIPTION OF THE DRAWINGS

For a greater understanding of the invention, and to show more clearly how it may be carried into effect, embodiments will be described, by way of example, with reference to the accompanying drawings, wherein:

FIGS. 1A and 1B are diagrams showing architecture of a scheme for generating an ECG from a subject's PPG, according to one embodiment; wherein E and P are original ECG and PPG signals, respectively, generated outputs are E′ and P′, reconstructed or cyclic outputs are E″ and P″, connections to the generators G are shown with solid lines, and connections to the discriminators D are shown with dashed lines.

FIG. 2 shows ECG signals generated by the embodiment of FIG. 1, wherein two different ECG signals are generated from each of the four ECG-PPG datasets (see the description).

FIGS. 3A-3D are attention maps wherein light areas indicate regions of ECG signals to which an attentive generator directs more attention compared to the darker regions; the four generated ECGs (A-D) correspond to different subjects.

FIGS. 4A-4C show three examples of ECGs generated from the corresponding PPG input, and the original ECG for comparison, obtained by paired training of the embodiment of FIG. 1.

FIGS. 5A-5C show three examples of ECGs generated from the corresponding PPG input that do not correspond to the original ECG signal.

### DETAILED DESCRIPTION OF EMBODIMENTS

There is a discrepancy between the need for continuous wearable ECG monitoring and the currently available solutions. Embodiments described herein address this discrepancy by providing a machine learning approach, such as a generative adversarial network (GAN) (Goodfellow et al. 2014), that takes PPG as input and generates an ECG. Embodiments may enable the system to be trained in an unpaired manner, and may be designed with attention-based generators and equipped with multiple discriminators. Attention mechanisms are used in the generators to better learn to focus on specific local regions such as the QRS complex of an ECG. To generate high fidelity ECG signals in terms of both time and frequency information, a dual discriminator strategy may be used where one discriminator operates on signals in the time domain while the other uses frequency-domain spectrograms of the signals. Results show that the generated ECG signals (e.g., PQRSTU waveforms) are very similar to the corresponding real ECG signals. Also, HR estimation was performed using the generated ECG as well as the input PPG signals. Comparing these values to the HR measured from the ground-truth ECG signals revealed a clear advantage in the embodiments.

As used herein, the term “signal” is intended to refer to a time series of data.

As described herein, a framework is provided for generating ECG signals from PPG signals. According to embodiments, attention-based generators and dual time and frequency domain discriminators together with an unpaired training method may be used to obtain realistic ECG signals. Although unpaired training has been proposed in the context of image synthesis (Zhu et al. 2017), no previous studies have attempted to generate ECG from PPG (or in fact any cross-modality signal-to-signal translation in the biosignal domain) using GANs or other deep learning techniques.

As described herein, a multi-corpus subject-independent study proves the generalizability of the embodiments to data from unseen subjects acquired under different conditions. The generated ECG provides more accurate HR estimation compared to HR values calculated from the original PPG, demonstrating benefits for the healthcare domain.

Embodiments may be implemented in a computer-readable medium. As used herein, “computer-readable medium” refers to non-transitory storage hardware, non-transitory storage device, or non-transitory computer system memory that may be accessed by a controller, a microcontroller, a microprocessor, a computer system, a module of a computer system, a digital signal processor (DSP), field-programmable gate array (FPGA), application-specific integrated circuit (ASIC), etc., generally referred to herein as a “processor”, having stored thereon computer-executable instructions (i.e., software programs, software code). Accessing the computer-readable medium may include the processor retrieving and/or executing the computer-executable instructions encoded on the medium. The non-transitory computer-readable medium may include, but is not limited to, one or more types of hardware memory, non-transitory tangible media (for example, one or more magnetic storage disks, one or more optical disks, one or more USB flash drives), computer system memory or random access memory (such as, DRAM, SRAM, EDO RAM) and the like.

Embodiments may be implemented in a computer-readable medium that is part of an electronic device or system, including a processor and one or more sensors, configured to provide measurement of a subject's heart rate, ECG, etc. The electronic device or system may be implemented as wearable on a subject's body, such as on an appendage, for example, wrist, ankle, finger. In various embodiments, the wearable electronic device may be configured as a wristwatch, a fitness device, or a medical device. The electronic device or system may be implemented with components (e.g., transmitters, receivers) that enable wired or wireless communications with each other, wherein at least one component is configured to be worn by a subject, and processing and data storage may be carried out at least partially on the wearable component. The electronic device or system may communicate with one or more remote servers and/or a cloud-based computing resource, wherein processing and/or data storage may be carried out at least partially on the one or more remote servers and/or a cloud-based computing resource. For such communications the transmitter/receiver may be configured to communicate with a network such as the Internet, a Wide Area Network (WAN), a Local Area Network (LAN), a cellular network, etc., to send data (for example sensor data, ECG data, etc.), based on established protocols/standards (e.g., utilizing one or more of radio frequency (RF) signals, cellular 2G, 3G, 4G, LTE, 5G, IEEE 802.11 standard such as WiFi, IEEE 802.16 standard such as WiMAX, Bluetooth™, ANT, ANT+, low energy (BLE), the industrial, scientific, and medical (ISM) band at 2.4 GHz, etc.). The electronic device or system may include an output device that provides the output ECG, for example, a display device that renders all or a part of the ECG signal, which may include all or any of PQRSTU features of the ECG.

The one or more sensors may include an optical sensor, such as one or more light emitters (e.g., LED) for emitting light at one or more selected wavelengths (e.g., infra-red (IR), green) toward the subject's skin, and one or more light detectors (e.g., photo-resistor, photo-transistor, photo-diode, etc.) for receiving light reflected from the subject's skin. The device or system may include an optical data processing module implemented in software, hardware, or a combination thereof for processing optical data resulting from light received at the light detector to provide PPG data used by the processor to determine the subject's ECG as described herein. Processing optical data may include combining with data from one or more motion sensors (e.g., accelerometer, gyroscope, etc.) to minimize or eliminate noise in the optical data caused by motion or other artifacts, or combining with optical data obtained at another wavelength.

The invention will be further described by way of the following non-limiting Example.

### EXAMPLE

### Objective and Architecture

In order to not be constrained by paired training where both types of data (ECG and PPG) are needed from the same instance in order to train the system, embodiments may be based on training using an unpaired GAN. Examples of an unpaired training approach include PPG and ECG signals obtained from the same subject at different times, or from different subjects. An objective of the embodiments is to learn to estimate the mapping between PPG (P) and ECG (E) domains. In order to force the generator to focus on regions of the data with significant importance, an attention mechanism is incorporated into the generator. The generator G_{E}: P→E was implemented to learn forward mapping, and G_{P}: E→P to learn the inverse mapping. The generated ECG and generated PPG were denoted as E′and P′ respectively, where E′=G_{E}(P) and P′=G_{P}(E). According to (Penttila et al. 2001) and a large number of other studies, cardiac activity is manifested in both time and frequency domains. Therefore, in order to preserve the integrity of the generated ECG in both domains, the use of a dual discriminator strategy was implemented, where D^{t} was employed to classify the time domain and D^{f} was used to classify the frequency domain response of real and generated data.

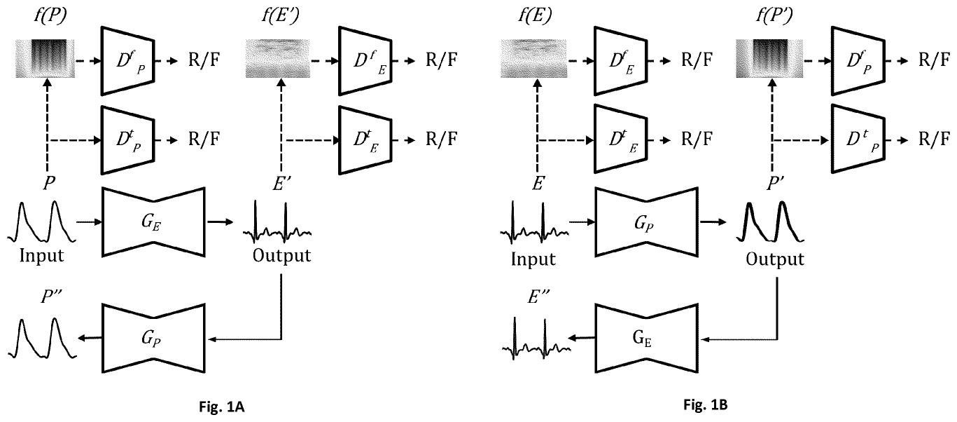

The diagrams of FIGS. 1A and 1B show the architecture of an embodiment, wherein each of FIGS. 1A and 1B show different connections during training. In FIGS. 1A and 1B, ECG (E) and PPG (P) are the original input signals, E′and P′ are the generated outputs, and E″and P″ are the reconstructed or cyclic outputs. Connections to the generators are marked with solid lines, whereas connections to the discriminators are marked with dashed lines. The embodiment of FIGS. 1A and 1B is implemented with four discriminators, two operating on the PPG data in the time and frequency domains, respectively, and two operating on the ECG data in the time and frequency domains, respectively.

Referring to FIG. 1A, G_{E} takes P as an input and generates E′ as the output. Similarly, in FIG. 1B, E is given as an input to G_{P} where P′ is generated as the output. In the embodiment D_{E}^{t} and D_{P}^{t} are employed to discriminate E versus E′, and P versus P′, respectively. Similarly, D_{E}^{f} and D_{P}^{f} are developed to discriminate f(E) versus f(E′), as well as f(P) versus f(P′), respectively, where f denotes the spectrogram of the input signal. Finally, E′and P′are given as inputs to G_{P} and G_{E} respectively, in order to complete the cyclic training process.

The dual discriminator, the feature of integrating an attention mechanism into the generator, and the loss functions used to train the overall architecture, and details and architectures of each of the networks used are described below.

### Dual Discriminators

As mentioned above, to preserve both time and frequency information in the generated ECG, a dual discriminator approach was used. To leverage the concept of dual discriminators, a Short-Time Fourier Transformation (STFT) was performed on the ECG/PPG time series data. Denote x[n] as a time-series, then STFT(x[n]) can be denoted as:

X(m, ω)=Σ_{n=-∞}^{∞}x[n]w[n−m]e^{-jωn} (1)

where m is the step size and w[n] denotes Hann window function. The spectrogram is obtained by f(x[n])=log(|X(m, ω)|+ϑ), where ϑ=1e⁻¹⁰ is used to avoid infinite condition. As shown in FIGS. 1A and 1B the time-domain and frequency-domain discriminators operate in parallel, and as will be discussed below, to aggregate the outcomes of these two networks, the loss terms of both of these networks are incorporated into the adversarial loss.

### Attention-Based Generators

Attention U-Net was used for the generator architecture, which has been recently proposed and used for image classification (Oktay et al. 2018; Jetley et al. 2018). Attention-based generators were chosen to learn to better focus on salient features passing through the skip connections. Assume x^{l} are features obtained from the skip connection originating from layer l, and g is the gating vector that determines the region of focus. First, x^{l} and g are mapped to an intermediate dimensional space R^{Fint} where Fᵢₙₜ corresponds to the dimensions of the intermediate-dimensional space. The objective is to determine the scalar attention values (αᵢ^{l}) for each temporal unit xᵢ^{l}∈ [symbol] ^{F}^{ₗ}, utilizing gating vector gᵢ∈R^{Fg}, where Fₗ and F_{g} are the number of feature maps in x^{l} and g respectively. Linear transformations are performed on x^{l} and g as ϑₓ=Wₓx^{l}ᵢ+bₓ and ϑ_{g}=W_{g}gᵢ+b_{g} respectively, where Wx∈RFl×Fint, Wg∈RFg×Fint, and bx, bg refer to the bias terms. Next, non-linear activation function ReLu (denoted by σ₁) is applied to obtain the sum feature activation f=σ₁(ϑₓ+ϑ_{g}), where σ₁(y) is formulated as max(0,y). Next a linear mapping of f onto the R_{Fint} dimensional space is done by performing channel-wise 1×1 convolutions, followed by passing through a sigmoid activation function (σ₂) in order to obtain the attention weights in the range of [0,1]. The attention map corresponding to x^{l} is obtained by αᵢ^{l}=σ₂(ψ*f) where σ₂(y) can be formulated as

$$\frac{1}{1 + {exp}^{- y}}$$

and * denotes convolution. Next, element-wise multiplication was performed between x^{l}ᵢ and αᵢ^{l} to obtain the final output from the attention layer.

### Loss

The final objective function is a combination of an adversarial loss and a cyclic consistency loss as presented below.

Adversarial Loss: Embodiments may apply adversarial loss in both forward and inverse mappings. Let's denote individual PPG segments as p and the corresponding ground-truth ECG segments as e. For the mapping function G_{E}: P→E, and discriminators D_{E}^{t} and D_{E}^{t}, the adversarial losses are defined as:

[symbol]

(G_{E}, D_{E}^{t})=E_{e˜E}[log(D_{E}^{t}(e))]+E_{p˜P}[log(1−D_{E}^{t}(G_{E}(p)))] (2)

[symbol]

(G_{E}, D_{E}^{f})=E_{e˜E}[log(D_{E}^{f}(f(e)))]+E_{p˜P}[log(1−D_{E}^{f}(f(G_{E}(p)))] (3)

Similarly, for the inverse mapping function G_{P}: E→P, and discriminators D_{P}^{t} and D_{P}^{f}, the adversarial losses are defined as:

[symbol]

(G_{P}, D_{E}^{t})=E_{p˜P}[log(D_{P}^{t}(p))]+E_{e˜E}[log(1−D_{P}^{t}(G_{P}(e)))] (4)

[symbol]

(G_{P}, D_{P}^{f})=E_{p˜P}[log(D_{P}^{f}(f(p)))]+E_{e˜E}[log(1−D_{P}^{f}(f(G_{P}(e)))] (5)

Finally, the adversarial objective function for the mapping G_{E}: P→E is obtained as:

$${min}_{{G}_{E}} {max}_{{D}_{E}^{t} {ℒ}_{𝒶𝒹𝓋}} ( {G}_{E} , {D}_{E}^{t} ) and {min}_{{G}_{E}} {max}_{{D}_{E}^{f} {ℒ}_{𝒶𝒹𝓋}} ( {G}_{E} , {D}_{E}^{f} ) . \qquad ( 6 )$$

Similarly, for the mapping G_{P}: E→P, can be calculated as:

$${min}_{{G}_{P}} {max}_{{D}_{P}^{t} {ℒ}_{𝒶𝒹𝓋}} ( {G}_{P} {,}_{P}^{t} ) and {min}_{{G}_{P}} {max}_{{D}_{P}^{f} {ℒ}_{𝒶𝒹𝓋}} ( {G}_{P} , {D}_{P}^{f} ) . \qquad ( 7 )$$

Cyclic Consistency Loss: The other component of the objective function is the cyclic consistency loss or reconstruction loss as proposed by (Zhu et al. 2017). In order to ensure that forward mappings and inverse mappings are consistent, i.e., p→G_{E}(p)→G_{P}(G_{E}(p))≈p, as well as e→G_{P}(e)→G_{E}(G_{P}(e))≈e, the cycle consistency loss minimization is calculated as:

[symbol]

(G_{E}, G_{P})=E_{e˜E}[∥G_{E}(G_{P}(e))−e∥₁]+E_{p˜P}[∥G_{P}(G_{E}(p))−p∥₁] (8)

Final Loss: The final objective function is computed as:

[symbol]

_{Final}=α [symbol] (G_{E}, D_{E}^{t})+α [symbol] (G_{P}, D_{P}^{t})+β [symbol] (G_{E}, D_{E}^{f})+β [symbol] (G_{P}, D_{P}^{f})+λ [symbol] (G_{E}, G_{P}) (9)

where α and β are adversarial loss coefficients corresponding to Dₜ and D_{f} respectively, and λ is the cyclic consistency loss coefficient.

### Experiments

### Datasets

Four popular ECG-PPG datasets were used, namely BIDMC (Pimentel et al. 2016), CAPNO (Karlen et al. 2013), DALIA (Reiss et al. 2019), and WESAD (Schmidt et al. 2018). These four datasets were combined in order to enable a multi-corpus approach leveraging large and diverse distributions of data for different factors such as activity (e.g., working, driving, walking, resting), age (e.g., children, middle-age, elderly, etc.), and others.

BIDMC (Pimentel et al. 2016) was obtained from 53 adult ICU patients (32 females, 21 males, mean age of 64.81) where each recording was 8 minutes long. PPG and ECG were both sampled at a frequency of 125 Hz.

CAPNO (Karlen et al. 2013) consists of data from 42 participants, out of which 29 were children (median age of 8.7) and 13 were adults (median age of 52.4). The recordings were collected while the participants were under medical observation. ECG and PPG recordings were sampled at a frequency of 300 Hz and were 8 minutes in length.

DALIA (Reiss et al. 2019) was recorded from 15 participants (8 females, 7 males, mean age of 30.60), where each recording was approximately 2 hours long. ECG and PPG signals were recorded while participants went through different daily life activities, for instance sitting, walking, driving, cycling, working and so on. ECG signals were recorded at a sampling frequency of 700 Hz while the PPG signals were recorded at a sampling rate of 64 Hz.

WESAD (Schmidt et al. 2018) was created using data from 15 participants (12 male, 3 female, mean age of 27.5), while performing activities such as solving arithmetic tasks, watching video clips, and others. Each recording was over 1 hour in duration. ECG was recorded at a sampling rate of 700 Hz while PPG was recorded at a sampling rate of 64 Hz.

### Data Preparation

Since the above-mentioned datasets were collected at different sampling frequencies, as a first step re-sampling (using interpolation) both the ECG and PPG signals (i.e., ECG and PPG data) was done with a sampling rate of 128 Hz. As the raw physiological signals contain a varying amounts and types of noise (e.g., power line interference, baseline wandering, motion artefacts), filtering techniques were applied to both the ECG and PPG signals. A band-pass FIR filter with a pass-band frequency of 3 Hz and stop-band frequency of 45 Hz were used on the ECG signals. Similarly, a band-pass Butterworth filter with a pass-band frequency of 1 Hz and a stopband frequency of 8 Hz was applied to the PPG signals. Next, person-specific z-score normalization is performed on both ECG and PPG. Then, the normalized ECG and PPG signals were segmented into 4-second windows (128 Hz×4 seconds=512 samples), with a 10% overlap to avoid missing any peaks. Finally, min-max [−1,1] normalization was performed on both ECG and PPG segments to ensure all the input data are in a specific range.

### Architecture

Generator: As mentioned earlier an Attention U-Net architecture was used as the generator, where self-gated soft attention units were used to filter the features passing through the skip connections. G_{E} and G_{P} take 1×512 data points as input. The encoder consisted of 6 blocks, where the number of filters gradually increased (64, 128 ,256, 512, 512, 512) with a fixed kernel size of 1×16 and a stride of 2. A layer normalization and leaky-ReLu activation was applied after each convolution layer except the first layer, where no normalization was used. A similar architecture was used in the decoder, except de-convolutional layers with ReLu activation functions were used and the number of filters gradually decreased in the same manner. The final output was then obtained from a de-convolutional layer with a single-channel output followed by tanh activation.

Discriminator: Dual discriminators were used to classify real and fake data in time and frequency domains. D_{E}^{t} and D_{P}^{t} take time-series signals of size 1×512 as inputs, whereas, spectrograms of size 128×128 are given as inputs to D_{E}^{f} and D_{P}^{f}. Both D^{t} and D^{f} use 4 convolution layers, where the number of filters gradually increased (64, 128, 256, 512) with a fixed kernel of 1×16 for D^{t} and 7×7 for D^{f}. Both networks use a stride of 2. Each convolution layer was followed by layer normalization and leaky ReLu activation, except the first layer where no normalization was used. Finally, the output was obtained from a single-channel convolutional layer.

### Training

An embodiment of the network based on the final objective function (equation (9) was trained on an Nvidia® Titan RTX™ GPU (Nvidia Corporation, Santa Clara, CA, USA), using TensorFlow™ (tensorflow.org). The aggregated ECG-PPG dataset was divided into a training set and test set. 80% of the users from each dataset (a total of 101 participants) were randomly selected for training, and the remaining 20% of users from each dataset (a total of 24 participants) for testing. To enable the embodiment to be trained in an unpaired fashion, ECG and PPG data from each dataset were shuffled separately eliminating the couplings between ECG and PPG followed by a shuffling of the order of datasets themselves for ECG and PPG separately. Adam optimizer was used to train both the generators and discriminators. In terms of hyperparameters, the model was trained for 15 epochs with a batch size of 128, where the learning rate (1e⁻⁴) was kept constant for the initial 10 epochs and then linearly decayed to 0. The values of α, β, and λ were set to 3, 1, and 30 respectively, although other values may be used. Other hyperparameters such as batch sizes (e.g., 16, 32, 64, 256, etc.), learning rates (e.g., 1e⁻³, 1e⁻⁵), epochs (e.g., 1 or more) may also be used.

### Performance

The embodiment produced two main signal outputs, generated ECG (E′) and generated PPG (P′). As the goal is to generate the more important and elusive ECG, E′is used and P′ is ignored in the following experiments. First the quantitative and qualitative results are presented. Next, an ablation study was performed in order to understand the effects of the different components of the model.

### Quantitative Results

Heart rate is measured as number of beats per minutes (BPM) by dividing the length of ECG or PPG segments in seconds by the average of the peak intervals multiplied by 60 (seconds). Define the mean absolute error (MAE) metric for the heart rate (in BPM) obtained from a given ECG or PPG signal (HR^{Q}) with respect to a ground-truth:

$$HR ( {HR}^{GT} ) as {MAE}_{HR} ( Q ) = \frac{1}{N} {\sum }_{i = 1}^{N} \vert {HR}_{i}^{GT} - {HR}_{i}^{Q} \vert$$

where N is the number of segments for which the HR measurements have been obtained. In order to investigate the merits of the embodiment, first measure MAE_{HR}(E′), where E′is the ECG generated by the embodiment. These MAE values are compared to MAE_{HR}(P) (where P denotes the available input PPG) as reported by other studies on the four datasets. The results are presented in Table 1 where it is observed that for 3 of the 4 datasets, the HR measured from the ECG generated by the embodiment is more accurate than the HR measured from the input PPG signals. For CAPNO dataset in which the ECG shows higher error compared to other works based on PPG, the difference is marginal, especially in comparison to the performance gains achieved across the other datasets.

Different studies in this area have used different window sizes for HR measurement which are reported in Table 1. To evaluate the impact of the model based on different window sizes, MAE_{HR}(E′) was measured over different 4, 8, 16, 32, and 64 second windows and the results are presented in comparison to MAE_{HR}(P) across all the subjects available in the four ECG-PPG datasets in Table 2. In these experiments, two algorithms were used for detecting peaks from ECG and PPG signals (Makowski et al. 2020). A clear advantage was observed in measuring HR from E′as opposed to P. There were consistent performance gains across different window sizes, which further demonstrates the stability of the results produced by the embodiment.

TABLE 1
Comparison of the MAE_{HR} calculated from the generated ECG
with MAE_{HR} calculated from the real input PPG.

| Dataset | Method | Window (sec) | MAE_{HR} |
|---|---|---|---|
| BIDMC | Nilsson et al. 2005 | 64 | 4.6 |
| | Shelly et al. 2006 | | 2.3 |
| | Fleming et al. 2007 | | 5.5 |
| | Karlen et al. 2013 | | 5.7 |
| | Pimentel et al. 2016 | | 2.7 |
| | Embodiment | | 0.7 |
| CAPNO | Nilsson et al. 2005 | 64 | 10.2 |
| | Shelly et al. 2006 | | 2.2 |
| | Fleming et al. 2007 | | 1.4 |
| | Karlen et al. 2013 | | 1.2 |
| | Pimentel et al. 2016 | | 1.9 |
| | Embodiment | | 2.0 |
| Dalia | Schäck et al. 2017 | 8 | 20.5 |
| | Reiss et at. 2019 | | 15.6 |
| | Reiss et al. 2019 | | 11.1 |
| | Embodiment | | 8.3 |
| WESAD | Schäck et al. 2017 | 8 | 19.9 |
| | Reiss et at. 2019 | | 11.5 |
| | Reiss et al. 2019 | | 9.5 |
| | Embodiment | | 8.6 |

TABLE 2
Comparison of MAE_{HR} between generated ECG and
real PPG for different window sizes.

| Window (s) | MAE_{HR}(E′) | MAE_{HR}(P) |
|---|---|---|
| 4 | 4.86 | 10.67 |
| 8 | 3.54 | 10.23 |
| 16 | 3.27 | 10.00 |
| 32 | 3.08 | 9.77 |
| 64 | 2.89 | 9.74 |

### Qualitative Results

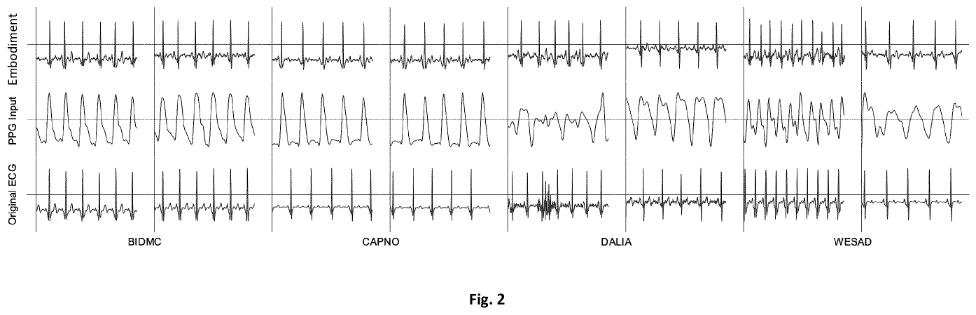

In FIG. 2 shows eight samples of ECG signals generated by the embodiment, wherein two different samples were generated from each of the four ECG-PPG datasets to better demonstrate the qualitative performance of the network. FIG. 2 clearly shows the network is able to learn to reconstruct the shape of the original ECG signals from corresponding PPG inputs. In some cases the generated ECG signals exhibit a small time lag with respect to the original ECG signals. The root cause of this time delay is the Pulse Arrival Time (PAT), which is defined as the time taken by the PPG pulse to travel from the heart to a distal site (from where PPG is collected, for example, wrist, fingertip, ear, or others) (Elgendi et al. 2019). Nonetheless, this time lag is consistent for all the beats across a single generated ECG signal as a simple offset, and therefore does not impact HR measurements or other cardiovascular-related metrics. This is further evidenced by the accurate HR measurements presented earlier in Tables 1 and 2.

### Ablation Study

Embodiments may include attention-based generators (Attn) and/or dual discriminators (DD), as discussed earlier. In order to investigate the usefulness of the attention mechanisms and dual discriminators, an ablation study of two variations of the network was performed by removing each of these components individually. To evaluate these components, the same MAE_{HR} was performed along with a number of other metrics, which are Root Mean Squared Error (RMSE), Percentage Root Mean Squared Difference (PRD), and Frechet Distance (FD). These are briefly defined as follows:
RMSE: To understand the stability between E and E′, calculate

$$RMSE = \sqrt{\frac{1}{N} {\sum }_{i = 1}^{N} {( {E}_{i} - {E}_{i}^{′} )}^{2}}$$

where Eᵢ and E′ⁱ refer to the i^{th} point of E and E′ respectively.
PRD: To quantify the distortion between E and E′, calculate

$$PRD = \sqrt{\frac{{\sum }_{i = 1}^{N} {( {E}_{i} - {E}_{i}^{′} )}^{2}}{{\sum }_{i = 1}^{N} {( {E}_{i} )}^{2}} \times 100} .$$

FD: Frechet distance (Alt and Godau 1995) is calculated to measure the similarity between the E and E′. While calculating the distance between two curves, this distance considers the location and order of the data points, hence, giving a more accurate measure of similarity between two timeseries signals. Let's assume E, a discrete signal, can be expressed as a sequence of {e₁, e₂, e₃, . . . , e_{N}} and similarly E′ can be expressed as {e′₁, e′₂, e′₃, . . . , e′_{N}}. A 2-D matrix M of corresponding data points can be created by preserving the order of sequence E and E′, where M⊆{(e, e′)|e∈E, e′∈E′}. The discrete Frechet distance of E and E′ is calculated as FD=min_{M}max_{(e,e′)∈M}d(e, e′), where d(e, e′) denotes the Euclidean distance between corresponding samples of e and e′.

The results of the ablation study are presented in Table 3, where the performance of different embodiments are shown for all the subjects across all four ECG-PPG datasets. The results show the benefit of using an embodiment with Attn and DD over ablation variants.

TABLE 3
Performance comparison of embodiments across
all subjects in the four ECG-PPG datasets.

| Embodiment | RMSE | PRD | FD | MAE_{HR} |
|---|---|---|---|---|
| without DD | 0.396 | 8.742 | 0.717 | 9.57 |
| without Attn | 0.386 | 8.393 | 0.773 | 9.67 |
| with DD and Attn | 0.364 | 8.356 | 0.694 | 4.77 |

### Analysis

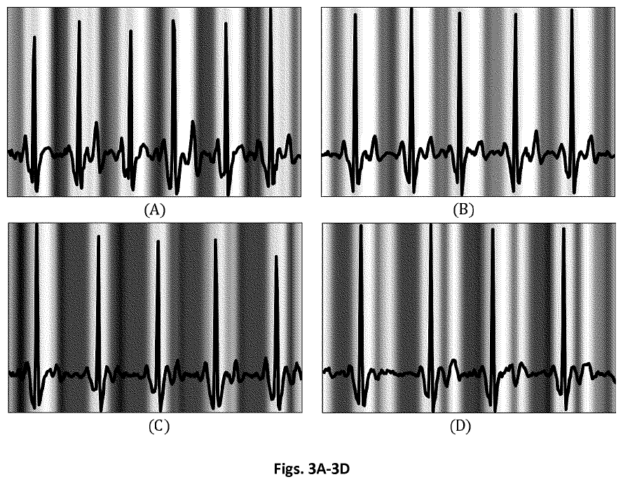

Attention Map: In order to better understand what was learned through the attention mechanism in the generators, the attention maps may be visualized as applied to the very last skip connection of the generator (G_{E}). The attention applied to the last skip connection was selected since this layer is the closest to the final output and therefore more interpretable. For better visualization, the attention map is superimposed on top of the output of the generator as in the examples of generated ECGs shown for four subjects in FIGS. 3A-3D. This shows that the model learns to generally focus on the PQRST complexes, which in turn helps the generator to learn the shapes of an ECG waveform better as evident from qualitative and quantitative results presented earlier.

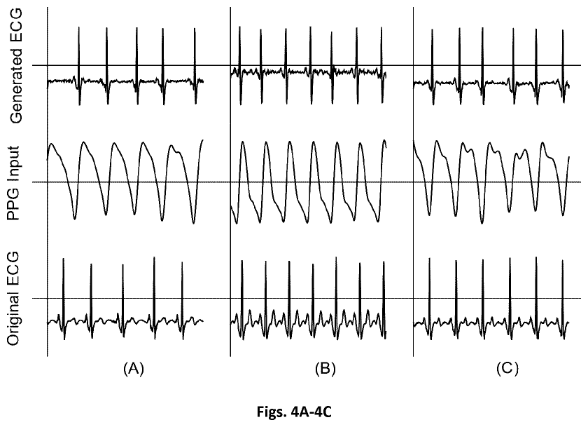

Unpaired Training vs. Paired Training: Performance of the embodiment with Attn and DD was investigated while training with paired ECG-PPG inputs in addition to the first approach which was based on unpaired training. To train the embodiment in a paired manner, the same training process mentioned above was followed, except coupling between the ECG and PPG pairs was maintained in the input data. The results are presented in Table 4, and three samples of generated ECGs are shown in FIGS. 4A-4C. By comparing these results to those presented in Table 4, it is observed that unpaired training shows superior performance compared to paired training. In particular, while paired training learns well to generate ECG beats from PPG inputs, it is less effective at learning the exact shape of the original ECG waveforms. This might be because an unpaired training scheme forces the network to learn stronger user independent mappings between PPG and ECG, compared to user-dependent paired training. While it can be argued that utilizing paired data in other GAN architectures might perform well, it should be noted that the goal here is to evaluate the performance when paired training is performed without any fundamental changes to the architecture. The embodiment was designed with the aim of being able to leverage datasets that do not necessarily contain both ECG and PPG, for example, use of the trained network in applications were only the PPG data is obtained. Hence, in unpaired training, datasets do contain both ECG and PPG signals are used so that ground truth measurements can be used for evaluation purposes.

TABLE 4
Results obtained using paired training.

| Method | RMSE | PRD | FD | MAE_{HR} |
|---|---|---|---|---|
| Paired | 0.437 | 9.315 | 0.748 | 5.04 |

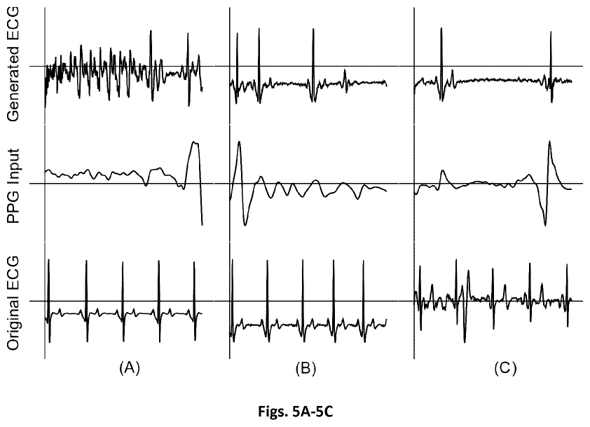

Failed Cases: There were instances where the embodiment failed to generate ECG signals that closely resembled the original ECG data. Such cases arise only when the PPG input signals are of very poor quality. Three examples are shown in FIGS. 5A-5C, wherein it can be seen that the PPG input signals were noisy and of poor quality.

### Applications and Demonstration

Apart from interest to the AI community, the methods and embodiments described herein have the potential to make a significant impact in the healthcare and wearable electronics domains, notably for continuous health monitoring. Monitoring cardiac activity is an essential part of continuous health monitoring systems, which could enable early diagnosis of cardiovascular diseases, detection of abnormal heart rhythms, and others, and in turn, early preventative measures that can lead to overcoming severe cardiac problems. Nonetheless, as discussed above, there are no suitable solutions for everyday continuous ECG monitoring. Methods and embodiments described herein bridge this gap by utilizing PPG signals (which can be easily collected from almost any wearable devices available) to capture cardiac information of users and generate accurate ECG signals. The multi-corpus subject-independent approach herein, where training data is from subjects engaged in a wide range of activities including daily life tasks, assures the embodiments are generally and widely applicable to all practical settings. Importantly, embodiments can be integrated into existing PPG-based wearable devices to extract ECG data without any required additional hardware. To demonstrate this concept, an embodiment (not described herein) has been implemented in a wrist-based wearable device that senses the wearer's PPG and uses the data to generate an accurate ECG signal. Applications may include generating multi-lead ECGs from PPG signals in order to extract more useful cardiac information often missing in single-channel ECG recordings. Furthermore, the approaches described herein open a new path towards cross-modality signal-to-signal translation in the biosignal domain, allowing for physiological recordings to be generated from readily available signals using more affordable technologies.

All cited publications are incorporated herein by reference in their entirety.

### EQUIVALENTS

While the invention has been described with respect to illustrative embodiments thereof, it will be understood that various changes may be made to the embodiments without departing from the scope of the invention. Accordingly, the described embodiments are to be considered merely exemplary and the invention is not to be limited thereby.

### REFERENCES

Alt, H.; and Godau, M. 1995. Computing the Frechet distance between two polygonal curves. International Journal of Computational Geometry & Applications 5(01n02): 75-91.
Ashley, E.; and Niebauer, J. 2004. Conquering the ECG. London: Remedica.
Bent, B.; Goldstein, B. A.; Kibbe, W. A.; and Dunn, J. P. 2020. Investigating sources of inaccuracy in wearable optical heart rate sensors. NPJ Digital Medicine 3(1): 1-9.
Elgendi, M.; Fletcher, R.; Liang, Y.; Howard, N.; Lovell, N. H.; Abbott, D.; Lim, K.; and Ward, R. 2019. The use of photoplethysmography for assessing hypertension. NPJ Digital Medicine 2(1): 1-11.
Fleming, S. G.; et al. 2007. A comparison of signal processing techniques for the extraction of breathing rate from the photoplethysmogram. Int. J. Biol. Med. Sci 2(4): 232-236.
Goodfellow, I.; Pouget-Abadie, J.; Mirza, M.; Xu, B.; WardeFarley, D.; Ozair, S.; Courville, A.; and Bengio, Y. 2014. Generative adversarial nets. In Advances in Neural Information Processing Systems, 2672-2680.
Jetley, S.; Lord, N. A.; Lee, N.; and Torr, P. 2018. Learn to Pay Attention. In International Conference on Learning Representations.
Karlen, W.; Raman, S.; Ansermino, J. M.; and Dumont, G. A. 2013. Multiparameter respiratory rate estimation from the photoplethysmogram. IEEE Transactions on Biomedical Engineering 60(7): 1946-1953.
Makowski, D.; Pham, T.; Lau, Z. J.; Brammer, J. C.; Lespinasse, F.; Pham, H.; Scholzel, C.; and S H Chen, A. 2020. NeuroKit2: A”Python Toolbox for Neurophysiological Signal Processing. URL https://github.com/neuropsychology/NeuroKit.
Nilsson, L.; et al. 2005. Respiration can be monitored by photoplethysmography with high sensitivity and specificity regardless of anaesthesia and ventilatory mode. Acta anaesthesiologica scandinavica 49(8): 1157-1162.
Oktay, O.; Schlemper, J.; Folgoc, L. L.; Lee, M.; Heinrich, M.; Misawa, K.; Mori, K.; McDonagh, S.; Hammerla, N. Y.; Kainz, B.; et al. 2018. Attention u-net: Learning where to look for the pancreas. arXiv preprint arXiv:1804.03999 .
Penttila, J.; Helminen, A.; Jartti, T.; Kuusela, T.; Huikuri, H. V.;”Tulppo, M. P.; Coffeng, R.; and Scheinin, H. 2001. Time domain, geometrical and frequency domain analysis of cardiac vagal outflow: effects of various respiratory patterns. Clinical Physiology 21(3): 365-376.
Pimentel, M. A.; Johnson, A. E.; Charlton, P. H.; Birrenkott, D.; Watkinson, P. J.; Tarassenko, L.; and Clifton, D. A. 2016. Toward a robust estimation of respiratory rate from pulse oximeters. IEEE Transactions on Biomedical Engineering 64(8): 1914-1923.
Reiss, A.; Indlekofer, I.; Schmidt, P.; and Van Laerhoven, K. 2019. Deep PPG: large-scale heart rate estimation with convolutional neural networks. Sensors 19(14): 3079.
Schmidt, P.; Reiss, A.; Duerichen, R.; Marberger, C.; and Van Laerhoven, K. 2018. Introducing wesad, a multimodal dataset for wearable stress and affect detection. In Proceedings of the 20th International Conference on Multimodal Interaction, 400-408.
Schäck, T.; et al. 2017. Computationally efficient heart rate estimation during physical exercise using photoplethysmographic signals. In 25th European Signal Processing Conference, 2478-2481. IEEE.
Shelley, K. H.; Awad, A. A.; Stout, R. G.; and Silverman, D. G. 2006. The use of joint time frequency analysis to quantify the effect of ventilation on the pulse oximeter waveform. Journal of clinical monitoring and computing 20(2): 81-87.
WHO. 2017. Cardio Vascular Diseases. https://www.who.int/news-room/fact -sheets/detail/cardiovascular-diseases-(cvds). (Accessed on Jul. 10, 2020).
Zhu, F.; Ye, F.; Fu, Y.; Liu, Q.; and Shen, B. 2019a. Electrocardiogram generation with a bidirectional LSTM-CNN generative adversarial network. Scientific Reports 9(1): 1-11.
Zhu, J.-Y.; Park, T.; Isola, P.; and Efros, A. A. 2017. Unpaired image-to-image translation using cycle-consistent adversarial networks. In Proceedings of the IEEE International Conference on Computer Vision, 2223-2232.
Zhu, Q.; Tian, X.; Wong, C.-W.; and Wu, M. 2019b. Learning Your Heart Actions From Pulse: ECG Waveform Reconstruction From PPG. bioRxiv 815258.

1. A method for generating an electrocardiogram (ECG) signal from a photoplethysmogram (PPG) signal, comprising:
receiving a PPG signal of a subject;
subjecting the PPG signal to a deep learning network trained to generate an ECG corresponding to the PPG signal; and
outputting the generated ECG signal.

2. The method of claim 1, wherein the deep learning network comprises a generative adversarial network (GAN) trained using unpaired PPG and ECG signals;
wherein the unpaired signals are obtained:
(a) from the same subject at different times; or
(b) from different subjects.

3. The method of claim 1, wherein the deep learning network comprises a generative adversarial network (GAN) trained using paired PPG and ECG signals;
wherein the paired signals are obtained from the same subject at the same time.

4. The method of claim 2 or 3, wherein the GAN comprises at least one generator and at least one discriminator.

5. The method of claim 4, wherein the at least one discriminator operates on ECG signals in the time domain.

6. The method of claim 2 or 3, wherein the GAN comprises at least one generator and first and second discriminators;
wherein the at least one generator translates the PPG signal to an ECG signal;
wherein the first discriminator operates on ECG signals in the frequency domain; and
wherein the second discriminator operates on ECG signals in the time domain.

7. The method of claim 2 or 3, wherein the GAN comprises first and second generators and first to fourth discriminators;
wherein the first generator translates the PPG signal to an ECG signal;
wherein the second generator translates the ECG signal to PPG signal;
wherein the first and second discriminators operate on ECG signals in the frequency and time domains, respectively; and
wherein the third and fourth discriminators operate on ECG signals in the frequency and time domains, respectively.

8. The method of claim 2 or 3, wherein at least one generator is an attention-based generator.

9. The method of claim 8, wherein the attention-based generator focusses on at least one selected region of the PPG and the generated ECG.

10. The method of claim 9, wherein the selected region comprises one or more of a P, Q, R, S, T, U component of the generated ECG.

11. The method of claim 1, comprising estimating heart rate (HR) using the generated ECG and the input PPG signal.

12. The method of claim 1, implemented in an electronic device.

13. The method of claim 12, wherein the electronic device is wearable.

14. An electronic device, comprising:
a processor that receives a PPG signal as an input;
wherein the processor implements a deep learning network trained to generate an ECG signal corresponding to the PPG signal; and
an output device connected to the processor that outputs the generated ECG signal.

15. The electronic device of claim 14, comprising a PPG sensor that obtains the PPG signal.

16. The electronic device of claim 14, wherein the deep learning network comprises a generative adversarial network (GAN) trained using unpaired PPG and ECG signals;
wherein the unpaired signals are obtained:
(a) from the same subject at different times; or
(b) from different subjects.

17. The electronic device of claim 14, wherein the deep learning network comprises a generative adversarial network (GAN) trained using paired PPG and ECG signals;
wherein the paired signals are obtained from the same subject at the same time.

18. The electronic device of claim 16 or 17, wherein the GAN comprises at least one generator and at least one discriminator.

19. The electronic device of claim 18, wherein the at least one discriminator operates on ECG signals in the time domain.

20. The electronic device of claim 16 or 17, wherein the GAN comprises at least one generator and first and second discriminators;
wherein the at least one generator translates the PPG signal to an ECG signal;
wherein the first discriminator operates on ECG signals in the frequency domain; and
wherein the second discriminator operates on ECG signals in the time domain.

21. The electronic device of claim 16 or 17, wherein the GAN comprises first and second generators and first to fourth discriminators;
wherein the first generator translates the PPG signal to an ECG signal;
wherein the second generator translates the ECG signal to PPG signal;
wherein the first and second discriminators operate on ECG signals in the frequency and time domains, respectively; and
wherein the third and fourth discriminators operate on ECG signals in the frequency and time domains, respectively.

22. The electronic device of claim 16 or 17, wherein at least one generator is an attention-based generator.

23. The electronic device of claim 22, wherein the attention-based generator focusses on at least one selected region of the PPG and the generated ECG.

24. The electronic device of claim 23, wherein the selected region comprises one or more of a P, Q, R, S, T, U component of the generated ECG.

25. The electronic device of claim 14, comprising estimating heart rate (HR) using the generated ECG and the input PPG signal.

26. The electronic device of claim 14 or 15, wherein the electronic device is adapted to be worn by a subject.

27. Non-transitory computer readable media for use with a processor, the computer readable media having stored thereon instructions that direct the processor to:
receive PPG signal of a subject;
implement a deep learning network trained to generate an ECG corresponding to the PPG signal;
subject the PPG data to the deep learning network; and
output the generated ECG signal.

28. The non-transitory computer readable media of claim 27, wherein the deep learning network comprises a generative adversarial network (GAN).

29. The non-transitory computer readable media of claim 28, wherein the GAN comprises at least one generator and at least one discriminator.

30. The non-transitory computer readable media of claim 29, wherein the at least one discriminator operates on ECG signals in the time domain.

31. The non-transitory computer readable media of claim 28, wherein the GAN comprises at least one generator and first and second discriminators;
wherein the at least one generator translates the PPG signal to an ECG signal;
wherein the first discriminator operates on ECG signals in the frequency domain; and
wherein the second discriminator operates on ECG signals in the time domain.

32. The non-transitory computer readable media of claim 28, wherein the GAN comprises first and second generators and first to fourth discriminators;
wherein the first generator translates the PPG signal to an ECG signal;
wherein the second generator translates the ECG signal to PPG data;
wherein the first and second discriminators operate on ECG signals in the frequency and time domains, respectively; and
wherein the third and fourth discriminators operate on ECG signals in the frequency and time domains, respectively.

33. The non-transitory computer readable media of claim 29, wherein at least one generator is an attention-based generator.

34. The non-transitory computer readable media of claim 33, wherein the attention-based generator focusses on at least one selected region of the PPG and the generated ECG.

35. The non-transitory computer readable media of claim 34, wherein the selected region comprises one or more of a P, Q, R, S, T, U component of the generated ECG.

36. The non-transitory computer readable media of claim 27, wherein the instructions direct the processor to estimate heart rate using the generated ECG and the input PPG signal.
